(19) United States
(12) Patent Application Publication
(10) Pub. No.: US 2015/0297173 A1
(43) Pub. Date: Oct. 22, 2015

(54) QUANTITATIVE TRANSMISSION ULTRASOUND IMAGING OF TISSUE CALCIFICATIONS

(71) Applicant: QT Ultrasound LLC, Salt Lake City, UT (US)

(72) Inventors: John C. Klock, Nicasio, CA (US); David T. Borup, Salt Lake City, UT (US); James W. Wiskin, Salt Lake City, UT (US); Elaine Iuanow, Danvers, MA (US)

(21) Appl. No.: 14/592,861

(22) Filed: Jan. 8, 2015

Related U.S. Application Data
(60) Provisional application No. 61/925,152, filed on Jan. 8, 2014.

(51) Int. Cl. A61B 8/08; A61B 8/14

(52) U.S. Cl. CPC A61B 8/085; A61B 8/14; A61B 8/5207; A61B 8/0825

(57) ABSTRACT

Methods and systems for imaging calcium in a subject are provided. In one aspect a method for imaging one or more micron-sized calcium deposits in a tissue of a subject can include delivering a transmission ultrasound wave field from a transmission transducer array to a body part of a subject, receiving transmission data from the transmission ultrasound wave field at a transmission receiver array, delivering a reflection ultrasound wave field from a reflection transducer array to the body part of the subject, receiving reflection data from the reflection ultrasound wave field at a reflection receiver array, generating speed of sound data from the transmission data, and refraction correcting the reflection data using the speed of sound data to generate a corrected reflection image showing a micron-sized calcium deposit image.

### PRIORITY DATA

This application claims the benefit of U.S. Provisional Patent Application Ser. No. 61/925,152, filed on Jan. 8, 2014, which is incorporated herein by reference.

### GOVERNMENT INTEREST

This invention was made with government support under The National Institute of Health Grant Nos. NCI 4R44CA110203-0022, 1R43CA123915-0, and 1RO1CA138536-01A2. The United States government has certain rights to this invention.

### BACKGROUND

Medical imaging is useful in the management of medical conditions for humans and animals. Such imaging can provide visual and functional information, thus allowing a medical provider to determine if a patient has a medical condition. Detailed imaging of bones and joints can be performed using a variety of technologies, including X-rays to create images on film, computerized tomographic X-ray imaging using solid state detectors (X-ray CT), magnetic resonance imaging (MRI), and hand-held ultrasound imaging. Each of these methods has its own particular advantages, disadvantages and limitations. X-ray based imaging, for example, can be disadvantageous due to the use of ionizing radiation that can cause tissue damage with each use (in some cases imperceptible, in other cases the cumulative dose of radiation can harm the patient). MRI imaging requires exposure to high magnetic fields, as well as claustrophobia (disqualifying up to 25% of patients), long imaging times, and high cost of the technology. Hand-held ultrasound imaging can beneficial due to portability and safety due to the use of non-ionizing radiation, as well as being less expensive than X-ray, CT, or MRI imaging. Hand-held ultrasound does, however, have limited resolution, and the quality of the diagnostic procedure is operator-dependent, with the best results requiring more highly skilled operators.

Deposits of calcium crystals can be found in tissues due to a number of conditions, including cancer, inflammation, infection, metabolic disease, degenerative conditions, aging, injuries, and the like. Large rounded calcium deposits (macrocalcifications) often occur in the breast of women over 50. They can often be seen as small white dots on a mammogram, and rarely need further testing.

Microcalcifications are smaller calcium deposits that can often be seen as tiny specks on a mammogram. These microcalcifications are not an indication of cancer in a majority of cases, but often need additional testing. Breast calcifications that are irregular in size and shape and/or are tightly clustered together are often considered suspicious. Suspicious calcifications may be associated with an invasive cancer, an intraductal cancer, or a pre-malignant disease, and as such may need further testing with imaging and/or tissue biopsy.

The detection of calcium deposits in tissues is often performed clinically using technologies that utilize ionizing X-ray radiation. Such ionizing radiation can be advantageous because calcium strongly absorbs X-ray energy, creating a “void” on the X-ray detector. This usually appears as a white spot on the X-ray image. Furthermore, this effect can be magnified by placing the X-ray detector farther from the calcium, however such magnification usually distorts the shape of the calcium deposit.

### BRIEF DESCRIPTION OF THE DRAWINGS

For a fuller understanding of the nature and advantage of the present disclosure, reference is being made to the following detailed description of various embodiments and in connection with the accompanying drawings, in which:

FIG. 1 shows a scanning system according to one aspect of the present disclosure.

FIG. 2 shows a scanning system according to one aspect of the present disclosure.

FIG. 3 shows a scanning system according to one aspect of the present disclosure.

FIG. 4 shows a top-down view of an imaging chamber and coupler device according to one aspect of the present disclosure.

FIG. 5 shows an illustration of a coupler device according to one aspect of the present disclosure.

FIG. 6 shows a flow diagram of a method for imaging calcium according to one aspect of the present disclosure.

FIG. 7 shows an image of balloon phantoms in tissue containing various amounts of calcium crystals QTUS imaged according to one aspect of the present disclosure.

FIG. 8 shows an image of balloon phantoms containing various amounts of calcium phosphate solutions QTUS imaged according to one aspect of the present disclosure.

FIG. 9 shows a different speeds for different human breast tissue types obtained while QTUS imaging normal human and cadaver breasts according to one aspect of the present disclosure.

FIG. 10 shows a coronal view image of a human subject's breast showing a deposit of calcium QTUS imaged according to one aspect of the present disclosure.

FIG. 11 shows a coronal view image of a human subject's breast showing a deposit of calcium around the rim of several oil cysts QTUS imaged according to one aspect of the present disclosure.

FIG. 12 shows a coronal view image of a human subject's breast showing deposits of calcium in the breast diagnosed as ductal carcinoma in situ QTUS imaged according to one aspect of the present disclosure.

FIG. 13 shows a coronal view image of a human subject's breast showing a large deposit of calcium in the breast QTUS imaged according to one aspect of the present disclosure.

FIG. 14 shows an image of results of using calcification parameters from the QTUS image of FIG. 13 to develop an interpretive image of the calcium of the breast of FIG. 10 according to one aspect of the present disclosure.

### DETAILED DESCRIPTION

Before the present disclosure is described herein, it is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting. Alterations and further modifications of the inventive features illustrated herein, and additional applications of the principles of the inventions as illustrated herein, which would occur to one skilled in the relevant art and having possession of this disclosure, are to be considered within the scope of the invention.

### DEFINITIONS

The following terminology will be used in accordance with the definitions set forth below.

It should be noted that, as used in this specification and the appended claims, the singular forms “a,” and, “the” include plural referents unless the context clearly dictates otherwise. Thus, for example, reference to “a transceiver” includes one or more of such transceivers and reference to “the logic processor” includes reference to one or more of such processors.

As used herein, “subject” refers to any mammal or other animal having at least bones and joints. Examples of subjects include humans, and may also include other animals such as horses, pigs, cattle, dogs, cats, rabbits, and aquatic mammals.

As used herein, “micron-sized” refers to structures or images of structures having a size of less than about 1000 microns. In one aspect, “micron-sized” can refer to structures or images of structures of having a size of less than about 1000 microns and greater than about 1 micron.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

The Disclosure

The present disclosure provides systems and devices for performing ultrasound imaging of calcium in tissues of a subject, including methods associated therewith. More specifically, such systems, devices, and methods utilize quantitative transmission ultrasound (QTUS) to image calcium in tissues. Such a system can be particularly useful for imaging not only the presence of calcium, but also in many cases the size, shape, and surface topography of deposits or other structural arrangements of calcium. Furthermore, QTUS imaging can detect tissue calcium with little to no distortion, thus rendering an accurate 3-D volume of calcium or a calcified cluster, mass, deposit, or other structure. Such a detailed level of calcium imaging can greatly impact the medical, veterinary, and research industries. For example, in the medical area of breast cancer, such a high level of calcium imaging can lower the incidence of tissue biopsies, and allow a more rapid diagnosis, either with imaging alone or with imaging and a subsequent tissue biopsy. A further advantage of QTUS imaging is that this detailed imaging of calcium can be performed without the use of ionizing radiation, and as such, is much safer for the subject and technicians performing the procedures. The present methods and systems can additionally generate images of such calcium structures that are free from artifacts such as, for example, ambiguous multiple images.

Calcium has a physical property that allows the absorption and scattering of sound energy in a unique way that can be different from other materials. This property of calcium can include not only selectively absorbing certain wavelengths of sound, but can also include converting that sound energy into a unique “harmonic” signal that can then be detected. In one aspect the inventors have developed unique processing techniques that can quickly and accurately model the generation of harmonics that are different from the fundamental frequency within the tissue. By delivering reflection mode ultrasound having a fundamental frequency of, for example, approximately 1.5 MHz, modeling the conversion of some of the acoustic energy to the 2nd associated harmonic (e.g. 3 MHz), and recording the received 2^{nd} harmonic signal, the high attenuation loss can be avoided that would normally prevent the reception of a meaningful acoustic signal if the original incident field were delivered at the 2^{nd} harmonic also. Furthermore, because the ultrasound signal has less distance to travel (e.g. approximately half as far), the attenuation effect is greatly reduced. The corresponding resolving power of the higher harmonic permits increased accuracy in the location, shape, topography, and/or clustering of the calcifications, thus greatly aiding the multivariate analysis, as well as the perspicuity of the calcification. Furthermore other tissues have this harmonics conversion property (measured by the so called “B over A” (B/A) parameter). Harmonics can be generated by the breast tissue and subsequently scattered from the calcium.

In another aspect of the present disclosure, the ultrasound beam can be configured to have certain beam characteristics that facilitate the detection of calcium in the tissue. The ability to program beam characteristics such as pulse timing, pulse duration, duty cycle, pulse energy (intensity), pulse carrier frequency or frequencies, and the like, can greatly facilitate the detection and characterization of calcium deposits in tissue.

As a general description, QTUS can be utilized to image, among other things, calcifications in tissues of a subject with great detail. In addition to increased soft tissue contrast and generally higher spatial resolution (500 microns as one non-limiting example) as compared to other imaging modalities, QTUS allows for a high degree of imaging energy manipulation from, for example, a programmable beam former. The beam former thus facilitates the ultrasound beam characteristic configurability described above. Furthermore, QTUS can utilize a fixed, mechanical device, and as such, resulting images are highly reproducible. Because the speed of the ultrasound energy through the body part can be accurately measured, the QTUS scanner can also provide quantitative data for each area of the tissue that is imaged. QTUS technology is also inherently less expensive to manufacture than other imaging systems such as X-ray, CT, or MRI, and can thus more affordably deliver high-resolution medical imaging. QTUS is additionally safer to use than X-ray, CT, or MRI, because it functions using non-ionizing radiation and does not require the injection of contrast media, although in various situations, contrast media can be used.

The inventors have discovered that ultrasound energy traversing through calcium deposits can be detected using inverse scattering algorithms, and as such, images of such deposits can be created. Thus, using QTUS as an imaging platform, high-resolution images of calcium deposits can surprisingly be obtained. Furthermore, the advantages of QTUS imaging over reflection ultrasound also include better soft tissue contrast and generally higher spatial resolution. QTUS imaging also provides for better quality 3D volume images and is highly reproducible. Because the speed of the ultrasound energy through tissue is accurately measured, the scanner also provides quantitative data for each area of the tissue that is imaged.

Without intending to be bound by any scientific theory, QTUS imaging can be used to detect calcium deposits through, in one exemplary aspect, refraction correcting reflection imaging data using speed of sound data derived from transmission mode imaging data. Transmission energy (or transmission data) received after passing through the tissue by transmission mode imaging enables the creation of a speed map (or speed data) of the voxels within an image, or within the space to which the resulting data is mapped. Reflection energy (or reflection data) received after reflection by the tissue enables the creation of a reflection image, or reflection data useable to create reflection image. By refraction correcting the reflection data, or in some cases a reflection image, calcifications can clearly be shown. In some cases, such calcifications are shown as “white” objects with high reflective values.

Additionally, while the speed of sound data can be used to correct for refraction, in some aspects the associated attenuation data can be used to create an automatically generated gain ramp for the reconstruction process. As such, in one aspect the reflection data can be refraction corrected using attenuation, or in other aspects, both speed and attenuation.

It is noted that any ultrasound signal or wave field design can be utilized that allows transmission mode imaging and reflection imaging, and any useful ultrasound signal characteristic design is considered to be within the present scope. In some aspects, however, short ultrasound pulses or “chirps” can be used in transmission mode imaging that contain, in some cases, multiple frequencies. In other aspects, large bandwidth pulses can be utilized for reflection mode imaging.

In addition to the quantitative tissue measurements (e.g. sound speed and attenuation), various morphometric parameters can be determined. In one non-limiting example for breast imaging, such morphometric parameters can include image descriptors similar to those in the Breast Imaging-Reporting and Data System (BI-RADS) breast imaging lexicon (shape, size, margins, shadowing, enhancement, etc.). Using univariate and multivariate methods, the diagnostic value can be appreciated and utilized of the various features obtained from different types of images, such as, for example, speed, attenuation, reflection tomography, and the like. The large number of parameters that can be evaluated can thus provide a very rich environment for diagnostic potential. Multivariate analysis can be used to identify, through analysis of the multivariate analysis training set, relevant parameters, including those parameters that are statistically collinear and therefore redundant.

Additionally, multivariate analysis can be used to determine various diagnostic sets of variables as predictors of various conditions in a subject, as generated from previous subject studies. Furthermore, once the calcification parameters are clearly outlined, an interpretative scheme for diagnostic interpretation of images obtained from HI the scanning system can readily be developed. In the case of breast imaging, for example, radiologists can utilize a workstation to independently review images and record reports of clinical results, including lesion descriptors for various image modalities. In some cases, an optimized classifier can be used to develop an interpretive scheme for diagnostic interpretation of images obtained from the QTUS system. It is additionally contemplated that the results from QTUS scanning can be used in a computer-aided diagnostic system.

As has been described, the present disclosure provides devices, systems, and methods for imaging calcium, calcifications, and/or calcium deposits in tissue of a subject. Calcium, calcifications, calcium deposits and like terms can be used interchangeably herein, and refer to any region, structure, mass, deposit, or other feature that include or are made up of calcium. In one aspect, QTUS protocol can be used to perform such imaging. Such scanning can capture images of calcium deposits in high detail. This can be particularly useful for imaging calcium deposits in breast tissue, however it is noted that the present disclosure is not limited to breast tissue, and that the present scope includes any body part or anatomical structure capable of being imaged accordingly. Furthermore, any size of calcium deposit that can be imaged using the disclosed technology is considered to be within the present scope. In one aspect, however, the calcium deposit or calcium deposit image can have an average size (or average diameter if appropriate), that is less than about micron-sized. In another aspect, the calcium deposit or calcium deposit image can have an average size of less than about 1000 microns. In another aspect, the calcium deposit or calcium deposit image can have an average size of from about 10 microns to about 1000 microns. In another aspect, the calcium deposit or calcium deposit image can have an average size of from about 30 microns to about 800 microns. In another aspect, the calcium deposit or calcium deposit image can have an average size of from about 60 microns to about 600 microns. In another aspect, the calcium deposit or calcium deposit image can have an average size of from about 100 microns to about 500 microns. In another aspect, the calcium deposit or calcium deposit image can have an average size of from about 50 microns to about 400 microns. In another aspect, the calcium deposit or calcium deposit image can have an average size of from about 30 microns to about 500 microns.

Such a system can be a non-invasive, diagnostic tool to provide detailed information about the physiology (i.e. bulk tissue properties) and anatomy (i.e. physical architecture) of the body part. In one aspect, the system can be used as an adjunct to X-ray, CT, MRI, PET, etc., imaging to aid physicians, veterinarians, and other medially oriented professionals in diagnosing conditions associated with calcium deposits by providing information about tissue properties that help to more clearly differentiate normal from abnormal, benign from malignant, or otherwise affected tissue in the body part. In another aspect, the system can replace other diagnostic testing, such as diagnostic X-rays, MRI, CT, PET, hand-held ultrasound, and other imaging technologies currently used. In yet another aspect the system can be used to provide real-time imaging for medical procedures, such as, for example, invasive medical procedures.

In general, the systems and methods according to aspects of the present disclosure can utilize ultrasound inverse scattering technology to produce a 3-D stack of tomography (2-D planar slice) images that can be similar or better in appearance and spatial resolution to CT or MR imaging methods. Direct 3-D imaging can be a further feature of the system. In one aspect, such images can be produced using two different techniques, namely Ultrasound Reflective Tomography (URT) and Ultrasound Inverse Scattering Tomography (UIST). Compared with conventional projection CT scans, URT images can be more detailed, easier to read, and are not obtained using potentially harmful ionizing radiation. Unlike conventional ultrasound, ultrasound images using inverse scattering technology completely penetrate and sample the entire body part, such as a breast for example, for increased uniformity and better overall resolution. In addition, such images are quantitative representations of ultrasound tissue properties, and therefore are not dependent on the system operator for image quality and consistency. The images can be reconstructed in three dimensions, thus providing an important visualization tool for diagnosis, biopsy, and surgery staging.

Various techniques are contemplated for refraction correcting the reflection data, and any such method or technique is considered to be within the present scope. In one aspect, for example, the refraction correction can be accomplished by modelling a beam (or a ray) that propagates according to an ordinary differential equation (ODE). As an example, the beam energy is followed along curved rays into the tissue, and the received signal energy can be mapped back into the tissue at the corrected location points. Additionally, the beam propagation can also be modeled as it actually occurs, i.e. as a wave front propagating through the heterogeneous tissue. This distinction can be relevant as the ‘beam’ so called is formed by time delays in neighboring elements, and the ‘beam’ is not in fact an infinitely thin ray. Also this ‘beam’ so called can be designed to be focused at successively further distances (dynamic focusing), but this focusing can be imperfect due to the very inhomogeneity in the tissue that causes refraction as well.

As such, in one aspect individual component data can be collected and the ‘beamforming’ process can be modeled. One exemplary method of accomplishing this modeling is performed using a ‘fast marching method’ for the solution of the partial differential equation (PDE) form of the eikonal equation. Note that there are two forms of the eikonal equation: the ODE form described above that is used to model idealized beams, and the PDE form that is a non-linear PDE that can be used to model the propagation of the wave front generated by the time delayed elements that surround the central element from which the ‘beam’ is assumed to propagate from.

As such, instead of modelling a single beam by the ordinary differential equation form of the eikonal equation, the wavefront as modeled by the partial differential equation form of the eikonal equation can be utilized. The ‘fast marching method’ is known in the literature, examples of which include “Shape Modeling with Front Propagation: A Level Set Approach”, Malladi, R., Sethian, J. A., and Vemuri, B. C. IEEE Trans; On Pattern Analysis and Machine Intelligence, 17, 2, pp. 158-175, 1995; and “a Fast Level set Method for Propagating Interfaces”, Adalsteinsson, D. and Sethian, J. A., J. Comp. Physics, 118, pp. 269-277, 1995, each of which is incorporated by reference in their entireties.

It is noted that, in some aspects, the reflection data can be collected from each component/element of the receiver array, that is, without using the beamforming capability. Such raw data can then be synthesized in software to dynamically focus at different depths, or synthetically focused to yield a reflection image.

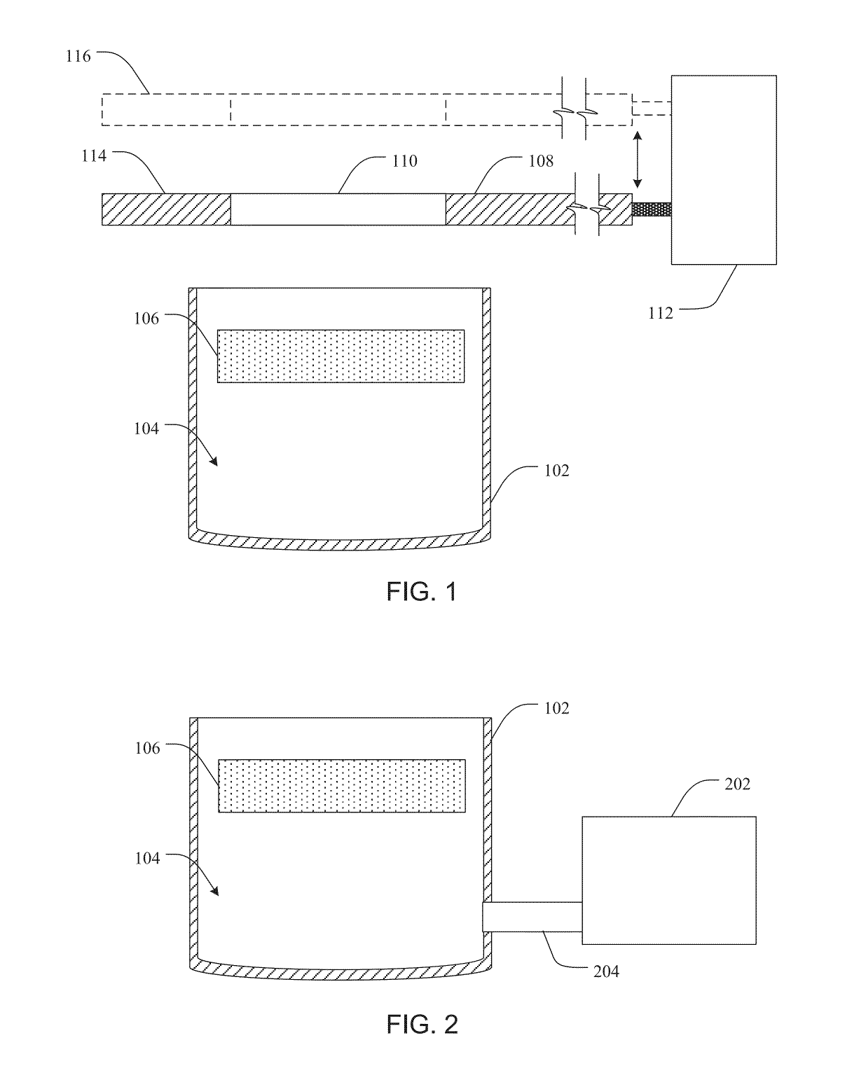

In one aspect, an ultrasound scanning system for imaging a body part of a subject is provided. In some aspects the body part can be a breast or other tissue that can develop calcium deposits. Such a system can include an imaging chamber operable to contain a HI liquid transmission medium and to receive a breast or other body part of a subject into the transmission medium, and at least one transducer array disposed in the chamber and operable to transmit and receive ultrasound signals within the medium. In one exemplary embodiment, as is shown in FIG. 1, an imaging chamber 102 is shown having an interior 104 into which a body part of a subject can be introduced (not shown). The interior 104 of the imaging chamber 102 can be filled with the transmission medium to facilitate the transmission of ultrasound therethrough. The transmission medium can be any liquid, including gels and jellies, or other material capable of deforming to receive a subject's body part therein that can facilitate the transmission of ultrasound through the chamber.

The imaging chamber can include a variety of configurations and be made from a variety of materials. It is noted that the imaging chamber structure and design should not be seen as limiting, and the details provided here are merely exemplary. In one aspect, however, the imaging chamber can be cylindrical, and in some aspects can be transparent. While the interior of the imaging chamber can be of any shape, a cylindrical design can be beneficial as it allows rotational motion of the arrays while minimizing volume. Transparent walls, while not necessary, allows the body part to be viewed during the scan, and allows an operator of the system to observe operation of the transducer arrays. Alternatively, the imaging chamber wall can be opaque or translucent, and in some aspects can have a window formed therein. The imaging chamber can also include fluidic devices forming inlet and/or outlet openings to allow fluid to enter and/or exit the bath. An upper end of the bath can be open to receive the body part, as described in greater detail below.

The imaging chamber 102 can also include at least one transducer array 106 disposed therein. The transducer array 106 can be positioned to deliver ultrasound energy into the transmission medium, and to receive at least transmission and/or reflection energy from the transmission medium. In one aspect, a transmission array can include one or more receiver operable to receive ultrasound signals and one or more transceivers operable to transmit ultrasound signals incorporated into a given array. Thus receivers and transceivers can be mixed in the same array. In another aspect, receivers and transceivers can be divided into separate arrays. It is additionally noted that in some aspects a transducer can act as a transceiver and as a receiver. Furthermore, in some aspects the transducer arrays can include transceiver and receiver elements that are positioned to interact with one another to generate an image.

The transducer arrays can be configured to move within the imaging chamber. For example, the transducer array(s) can move within the chamber to obtain both reflection and transmission information used to generate images and diagnostic information. Such arrays can be designed to rotate, as well as move up and down to generate a complete 3-D data set for the area of interest or even for the entire body part. In general, ultrasound pulses can be used for two imaging modalities: reflection and transmission. For refection images or data, the system emits a signal from one array and receives the reflected energy back in the same array or a different array that is positioned to receive such reflections. For transmission images or data, the system emits a signal from one array and receives the transmitted energy through the body part at a separate array, or at the same array that has been moved into an appropriate position prior to receiving the transmitted pulse. As one example, an array collecting reflection images or data can emit a pulse at a variety of positions around the body part. The number of incremental positions can vary depending on the design of the device and the imaging procedure, however in one non-limiting example, the array can emit pulses at 20 positions (every 18 degrees) around the body part. As with the reflective image case, an array collecting transmission images or data can emit a pulse at a variety of positions around the body part, and the number of incremental positions can vary depending on the design of the device and the imaging procedure. In one non-limiting example, however, during the same rotation sequence described for the reflection data, an array generating transmission images can emit an ultrasound signal into and through the body part at 180 different locations (every 2 degrees) around the entire body part to be received by an opposing transmission receiver. This allows the system to simultaneously generate data for both reflection and transmission sound properties of the body part. Alternatively, the arrays can move and/or emit continuously, and in some aspects the reflection and transmission arrays can generate images independent of one another, such as, for example, rotations that are exclusive for reflection or rotations that are exclusive for transmission.

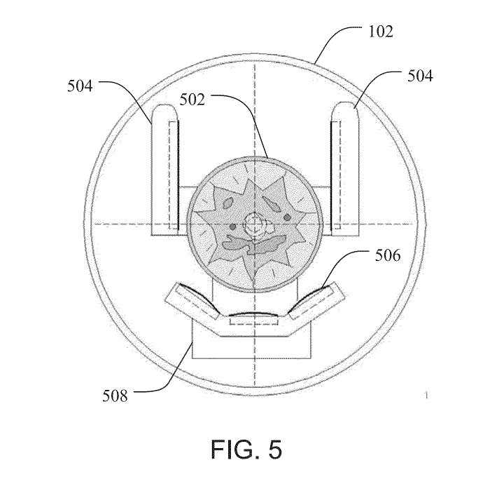

In one specific non-limiting aspect, a system can utilize at least two ultrasound arrays that rotate around the body part to generating true 3-D images and diagnostic information in a commercially viable timeframe, such as less than about 20 minutes per exam. The system can include two opposing ultrasound transducer arrays, as is shown in FIG. 5, movably disposed in the imaging chamber 102 to obtain both reflection and transmission data used to generate images and diagnostic information. The arrays are mechanically designed to rotate and move up and down generating a complete 3-D data set for the area of interest or even for the entire body part 502. In the case of FIG. 5, the system includes two opposing transmission arrays 504, and a reflection array 506 that can function as described above.

In some aspects, an imaging system can produce three separate images using two different imaging techniques: 1) transmission data generates images representing bulk tissue properties of speed of sound and attenuation of sound at each point in the body part; and 2) data generated from reflection information generates detailed reflective tomographic images, which in some cases can be refraction corrected using, for example, speed of sound data. These imaging techniques can be combined to effectively produce a three-dimensional stack of “slices” of the body part. Data from the ultrasound source is analyzed, and a quantitative map of tissue properties is rendered. As has been described above, in transmission mode the energy propagates through the tissue. In reflection mode, the energy reflects back from the tissue to the receivers. In both cases, the energy of the acoustic wave is refracted and scattered from the tissue it encounters. In this process multiple physical phenomena can take place: reflection, refraction, diffraction, multiple scattering events, and the like. These effects are generally ignored in present ultrasound, and as such, resulting images are significantly degraded, therefore rendering traditional ultrasound useful only in differentiating architectural or structural properties within the body part. In present ultrasound imaging system it is impossible to acquire quantitative values at a level sufficient for diagnosis of tissue characteristics using standard reflection ultrasound or imaging. Further details of transmission and reflection technology and imaging are disclosed in U.S. Pat. Nos. 4,662,222; 5,339,282; 6,005,916; 5,588,032; 6,587,540; 6,636,584; 7,570,742; 7,684,846; 7,699,713; 7,771,360; 7,841,982; 8,246,543; and 8,366,617, which are incorporated herein by reference in their entireties.

As one example configuration, transducer arrays can be disposed in the imaging chamber and carried by an armature, also disposable in the chamber. The armature can include a u-shaped member disposed on a vertical column that extends through a bottom of the imaging chamber. Each vertical arm of the u-shaped member can, for example, carry one of the arrays shown in FIG. 5. The u-shaped member can be sized to position the arrays around the body part. The arrays and can be rotatable around an axis of rotation, and displaceable vertically. For example, the armature can rotate around the vertical column, thus rotating the arrays. A rotational motor can be coupled to the armature to rotate the armature. For example, the rotational motor can be a rotational step motor coupled to the armature or vertical column by a belt. In addition, a linear motor can be coupled to the armature to linearly displace the armature, and thus the transducer arrays. For example, the vertical column can be carried by a platform on a plurality of rods. One of the rods can be threaded. The linear motor can engage the threaded rod such that rotation of the motor can raise and lower the platform, and thus the vertical column along with the rotational motor. A rotational and/or sliding seal can be formed between the imaging chamber and the armature, or vertical column, to seal the imaging chamber where the armature or vertical column passes through the bottom of the chamber. In addition, one or more bearings or rotational bearings can be disposed between the vertical column and the platform to facilitate rotation and reduce frictional forces. Thus, the platform can carry the armature and related motors to move the armature.

In addition, in some aspects the arrays can be tilted, or rotatable to have tilted orientation to allow imaging closer to an area of interest. For example, the arrays can be angled or directed in an upwardly angled direction so that the arrays emit upwardly at an angle and receive downwardly at an angle. Furthermore, the transducers can be disposed around the body part, and along the length of the body part, so that the transducers do not have to be moved or rotated.

In some aspects, transducer arrays can be incorporated into a fixed ring, or a portion of a ring. The ring can also be offset or non-concentric. The transducer arrays can send and receive ultrasound signals at a plurality of elevational locations along the body part, and at a plurality of rotational orientations around the body part at each elevational location. A linear motor can move the transducer arrays sequentially through a plurality of different elevational locations along the body part. Array elements can independently emit pulses at a number of distances from each other (depending on the number of elements in the circle) around the body part. During the same pulsing sequence, the transmitting elements or transducers can emit an ultrasound signal into and through the body part at many different locations (depending again on the density of array elements around the ring) around the entire body part. In one aspect, resulting transmission waveforms can be received by the opposing array elements. In another aspect, resulting reflection waveforms can be received by adjacent array elements, substantially adjacent array elements, or other elements in sufficiently close proximity to receive reflection energy. The arrays can then be moved to a different location along the body part and the sequence repeated. Alternatively, the arrays can emit during a continuous motion. Thus, the movement of the arrays and armature can be discrete, or stepwise through discrete position, or continuous. Additionally, the ring array can be tilted as described above.

It is additionally contemplated that a transducer array can be configured and sized to match a give subject and/or a given body part. Sizing the aperture of the array to the body part can result in greater comfort to the subject, and can allow a maximum amount of the body part to extend through the aperture and possibly better image quality.

Returning to FIG. 1, in another aspect the system can include a substantially horizontal table 108 disposable over the imaging chamber 102 and operable to support the subject thereon. It is noted that in some aspects a table or other support device for a subject can be oriented in any direction, from vertical to horizontal. An aperture 110 in the table can be positioned over the imaging chamber 102 such that a body part, such as a breast, of the subject can be passed therethrough and into the imaging chamber 102. A movement actuator 112 can be coupled to the table 108 to allow the table 108 and the imaging chamber 102 to be vertically displaceable with respect to one another. In some aspects, the movement actuator can be coupled, either directly or indirectly, to the imaging chamber. In yet another aspect, the movement actuator 112 can be operable to maintain the table over the imaging chamber in 1) a first position 114 whereby the table is sufficiently proximal to the imaging chamber to allow a body part of the subject to be within the imaging chamber when the subject is supported by the table, and 2) a second position 116 whereby the table is sufficiently distant from the imaging chamber to allow a body part of the subject to be suspended above the imaging chamber when the subject is supported by the table.

The system can also optionally include a securing device that is operable to secure the body part of the subject in the imaging chamber. Such a device can function to minimize movement of the subject during imaging, as well as providing comfort, such as, for example, a support rest that allows the subject to maintain a body position for a period of time. The securing device can be located at any position that is beneficial for the imaging procedure, and can vary depending on the design of the system and the body part of the subject. In some aspects the securing device can be located above the imaging chamber, such as, for example, coupled around a table aperture. In other aspects the securing device can be positioned within the interior of the imaging chamber, on top of the imaging chamber or otherwise affixed to the outside of the imaging chamber. In other aspects the securing device can include multiple securing sections that may or may not be coupled together in the same device. For example, a securing device may have a first securing section that is located below the transducer arrays in the imaging chamber and a second securing section that is located above the transducer arrays either in or above the imaging chamber.

In some aspects the system can include a variety of fluidic devices and/or components to process and handle the transmission medium. It is noted that the various components can vary depending on the design of the system and the nature of the transmission medium. In one aspect, as is shown in FIG. 2 for example, the system can include a conditioning chamber 202 fluidly coupled to the imaging chamber 102. The conditioning chamber can be used for a variety of conditioning tasks, such as thermal regulation of the transmission medium, cleaning of the transmission medium and/or the conditioning chamber, osmotic balance, liquid motion control, and the like, including appropriate combinations thereof. As such, the conditioning chamber can be utilized to condition the transmission medium before, during, and/or after an imaging procedure. Additionally, in some aspects conditioning or at least a portion of the conditioning can occur on the transmission medium in the conditioning chamber, while in other aspects the conditioning or at least a portion of the conditioning can occur on the transmission medium in the imaging chamber. The fluidic coupling between the imaging chamber 102 and the conditioning chamber 202 can be any fluidic coupling mechanism known. In one aspect, the system can include a fluidic system 204 operable to transfer the transmission medium from the conditioning chamber 202 to the imaging chamber. In some aspects the fluidic system 204 can also operate to transfer the transmission medium either back into the conditioning chamber or to another chamber or to a waste or recycling system.

As such, a portion of the body of a subject can be place into the imaging chamber for an imaging procedure. The imaging chamber can then be filled with the transmission medium, either prior to, during, or following the introduction of the body part into the chamber. The transmission medium is operable to transmit ultrasound energy. Because the transmission medium is coupled to the at least one transducer array and the body part, ultrasound energy is efficiently transmitted therebetween. It is noted for this and subsequent figures, the use of callout numbers used in previous figures denotes the same or similar element of that previous figure, and that the description of that element should be incorporated into the present figure where appropriate.

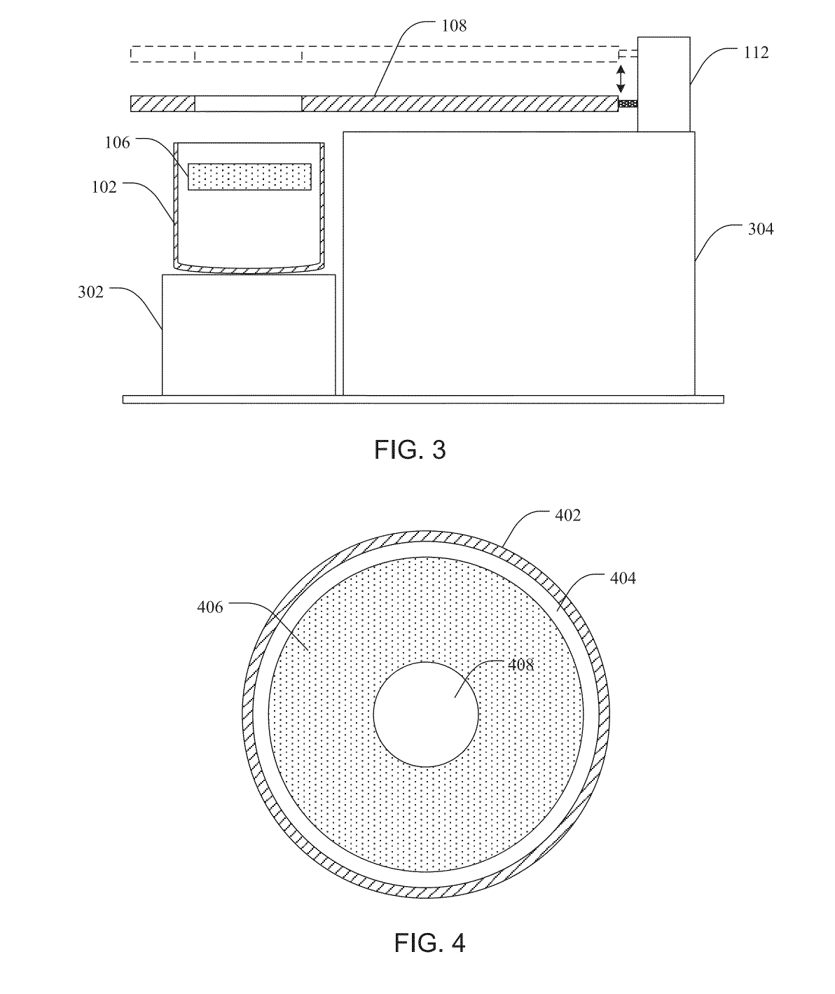

In another aspect, as is shown in FIG. 3, the system can include an imaging chamber support 302 for supporting the imaging chamber 102. The imaging chamber support 302 can be operable to perform a variety of functions in addition to merely supporting the imaging chamber 102. Non-limiting examples include 1D, 2D, 3D, and/or rotational movement of the imaging chamber 102, 1D, 2D, 3D, and/or rotational movement of the at least one transducer array, communication coupling between the at least one transducer array and any computer or other electronic device sending or receiving data or other signals from the transducer array, thermal regulation of the imaging chamber 102 and/or the transmission medium, and the like, including appropriate combinations thereof.

Furthermore, in some aspects the system can include a housing 304 capable of providing support and substance to the system, as well as containing various system elements. For example, in one aspect the housing 304 can provide support to the movement actuator 112 and the table 108. The housing 304 can be continuous or discontinuous with the imaging chamber support 302. Furthermore, in some aspects various ultrasound generation and/or ultrasound analysis systems, including various communication networks associated therewith, can be incorporated either fully or partially into the housing 304, and in some cases into the image chamber support 302.

In some aspects, the system can also include a light source (e.g., a laser pointer) to project a light beam (such as a fan beam) onto the body part at an area of interest. The area of interest can be marked prior to immersing the joint into an image chamber or into a ring array. The area of interest can be determined beforehand by reference to body part examinations, X-rays, etc. In one aspect, the light source can be mounted to the armature, and can be positioned at the arrays. Thus, the armature and arrays can be raised or lowered until the light beam from the laser pointer aligns with the mark on the body part corresponding to the area of interest. This position can be saved in the system as a center of the area of interest, and the scan can begin and end at a predetermined distance above and below the center of the area of interest. It will be appreciated that the position of the armature, and thus the arrays, can be determined from the motors used to position the armature, or from other sensors.

In addition, a camera can be positioned to provide an image of the body part and the arrays. The camera can be coupled to the system and/or a display or control module associated with the system. The camera can be mounted on the armature or ring array and positioned thereby. A horizontal line, or cross-hair, can be provided on the display, camera, or system to align the camera, and thus the arrays, with the mark on the body part corresponding to the area of interest. The camera can also include a light source, such as, for example, one or more lights, LEDs, or the like.

The various systems, devices, and methods of the present disclosure can also utilize an ultrasound transmission coupler in lieu or in addition to a liquid transmission medium where appropriate. For example, a sleeve or other configuration of coupler can be positioned around or in contact with a region of the subject to be imaged. In other words, the coupler can surround the region of the subject in some cases, and the coupler can contact only a portion of the region in other cases. The coupler thus facilitates the transmission of ultrasound energy between the transducer array and the subject, and as such the design and placement of the coupler can vary depending on the desired use, the size and shape of the body part, and the like. In some aspects a liquid, semiliquid, gel, or other like substance can be introduced between the transducer array and the coupler and/or between the coupler and the subject to improve ultrasound transmission therebetween. Additionally, in some aspects the coupler device can have a fixed shape and material composition, while in other aspects the coupler device can be a reservoir or can contain a reservoir that can be filled with a transmission medium that can transmit ultrasound. Such a fillable coupler device can conform to the shape of the body part, and thus may improve ultrasound transmission.

Accordingly, in one aspect an ultrasound scanning system for imaging calcium deposits in a tissue of a subject can include an imaging chamber operable to receive and contact a coupler device engaging a region of a body part of the subject, and at least one transducer array. In one aspect the at least one array of transducers can be positioned to at least partially encircle the coupler device. As such, a portion of the body of a subject is coupled to a coupler device that is operable to transmit ultrasound energy. Because the coupler device is coupled between the at least one transducer array and the body part, ultrasound energy is efficiently transmitted therebetween. The coupler device can surround the body part, a portion of the body part, or the coupler device can partially surround the body part or portion of the body part. The coupler device can also merely contact a portion of the body part sufficient to transmit ultrasound between the transducer array and the body part. Additionally, in some aspects the transducer array can be a ring of transducer arrays configured to couple to and/or to surround the coupler device.

In one aspect, as shown in FIG. 4, a system can include an imaging chamber 402 and at least one transducer array 404 positioned within or otherwise associated with the imaging chamber 402. FIG. 4 shows a circular transducer array, however it is contemplated that the imaging chamber can include multiple transducer arrays and/or non-circular transducer arrays. A coupler device 406 is positioned within the imaging chamber 402 to facilitate transmission of ultrasound energy between the transducer array 404 and the body part of the subject that is positioned within a receiving cavity 408 of the coupler device 406.

The design of a coupler device can vary widely depending on the body part being imaged and the design of the imager. In some aspects the coupler can be a wrap-around device. In other aspects, the coupler can be fashioned as a receptacle to receive the body part. In yet other aspects, the coupler can be formed to fit a given subject. As has been described, it is additionally contemplated that in some aspects the coupler can include a reservoir that is finable with a transmission medium. Furthermore, in some cases the coupler device can be placed into an imaging chamber for imaging. In other cases transducer arrays can be positioned around the coupler device or incorporated into the coupler device. Such a design can be beneficial for use in a mobile imaging system.

In one aspect, an ultrasound scanning system for imaging micron-sized calcium deposits in a tissue of a subject is provided. Such a system can include a transmission transducer array, and a transmission receiver array positioned to receive ultrasound energy transmitted through a body part and delivered from the transmission transducer array. The system can further include a reflection transducer array and a reflection receiver array positioned to receive ultrasound energy reflected by the body part and delivered from the reflection transducer array. It is noted that the transmission arrays and receiver arrays can be distinct or can be different functionality of the same array, for both the transmission and reflection modalities, as well as shared modalities between transmission and reflection. For example, in one aspect a single transducer array can function as both a transmission and reflection transducer array. In such a case, the transmission and reflection receiver arrays can be separate arrays that each receive signal or data from the transducer array, or the transmission array and the reflection array can be different functionalities of the same physical receiver array. It is to be understood that any combination of transmitter array and receiver array components and/or modalities, for both transmission and reflection, is considered to be within the present scope. As such, the transducer array 106 in FIG. 1 is intended to be a simplified representation of the various combinations of transmitting and receiving arrays for both transmission and reflection modalities.

In another aspect, the system can additionally include a computation system functionally coupled to at least the transmission receiver array and to the reflection receiver array, which is operable to generate speed of sound data from the transmission data, and to process the reflection data using the speed of sound data to generate a corrected reflection image. A beam former can additionally be included in the system that is functionally coupled to at least one of the transmission transducer array or the reflection transducer array.

While numerous configurations and designs of the computational system are contemplated, in one aspect such a system can include a nontransitory computer readable medium functionally coupled to the transmission receiver array and to the reflection receiver array, which is operable to receive and store transmission data and reflection data. A computational module can additionally be coupled to the nontransitory computer readable medium that is operable to generate speed of sound data from the transmission data, and to process the reflection data using the speed of sound data to generate a corrected reflection image. In some aspects, the computational module can be a computational processor, while in other aspects the computational module can be a larger system or grouping of systems to perform computational processes.

It should be understood that numerous system configurations of the ultrasound generation, delivery, capture, and analysis are contemplated, and that any such configuration is considered to be within the present scope. In one aspect, for example, an architecture or platform can include custom boards to provide data rates fast enough to store and process the amount of data gathered by the arrays. The architecture of the boards can vary depending on the design, provided the boards can operate at sufficiently fast data rates.

To render the images of the body part there is a complex series of image processing steps that can be used. One practical method uses frequency domain data, and a parabolic approximation to the full Helmholtz equation. This yields a very fast forward problem solver, the concomitant Jacobian is straightforward, and the desired (see below) fast implementation of the adjoint of the Jacobian calculation can be carried out after some algebraic manipulation. The mathematics associated with various processing steps are described in further detain in U.S. Pat. Nos. 5,588,032; 6,005,916; 6,587,540; 6,636,584; 7,570,742; 7,684,846; 7,841,982; and 8,246,543, each of which are incorporated herein by reference in their entireties.

Other implementations of parabolic approximation can additionally be utilized, for example those that utilize an averaged background speed of sound, two-way propagating fields, or the like.

The following description is one exemplary embodiment of a hardware/software system that can be utilized to gather and process data from an image scanner. In some aspects, components of the platform can be used to control the ultrasound delivery and the mechanical manipulation of the transducer arrays. It is noted that as the technological level of computer systems increase over time, it is understood that the architecture of the present hardware/software platform can change as well. The present scope is not limited by the current state of computer technology, and it is understood that advances in computer technologies are considered to be within the present scope.

In one non-limiting example, the architecture of a useful hardware/software platform can resemble a Linux cluster super-computer. The cluster can include five or more nodes interconnected at high speed, such as by, for example, a high-speed Ethernet network. In addition, each node can have access to a shared file system. Two or more of the nodes can be configured as Compute Nodes. Each Compute Node can include a

Single Board Computer (SBC) and a Fibre Channel Host Adapter (FCHA). Two of the nodes can be configured as data acquisition nodes. Each Data Acquisition Node can include a SBC, FCHA, Waveform Generator Card, Data Acquisition Cards, and Mux Cards. The FCHA can connect the SBC to the global file system.

An exemplary Compute Node can include a SBC, FCHA and CompactPCl backplane that the two cards plug into. One or more of the backplanes can be installed in a 19 inch rackmount chassis. The chassis provides power and cooling to the cards plugged into the backplanes.

The design and components of an SBC can vary depending on the computational level of a given imaging process and the desired speed of data processing. Generally each SBC can include a CPU, volatile memory (e.g., 16 GB of SDRAM), an interface to the Compact PCI backplane, and an onboard PCI bus. The PCI bus can support any number of peripherals, depending on the design of the system and the preferences of the designer and/or operator. Non-limiting examples of such peripheral/peripheral devices can include mice, keyboards, parallel ports, data storage interfaces, USB, networking, and video controllers. The data storage interface can include any include any type of interface, including, for example, Integrated Drive Electronics (IDE), Enhanced IDE (EIDE), ATA, Serial ATA (SATA), and the like. Non-limiting examples of networking interfaces include wireless cards for Wi-Fi networking, Bluetooth interfaces, 10/100/1000 Ethernet cards, fiber optic communication, and the like. Video controllers can vary widely, and can include any video card that can process at a desired level given the image processing operations. As such, in one aspect the video card or cards can be PCI based, and can interface with the PCI bus as described. In other aspects the SBC can include a dedicated video card bus such as, for example, PCI Express, Accelerated Graphics Port (AGP), and the like. In some aspects, the SBC can include multiple video processing cards. Additionally, in other aspects the SBC can include onboard video processing.

As with many computer components, CPUs can vary widely and are constantly increasing in computational power over time. The CPU used in the SBC can vary across image scanning and processing systems, and can also vary within a given system. For example, a system used in medical diagnostic procedures may utilized all high end CPUs in order to process images rapidly to facilitate diagnosis. Also, in situations where the scanning system is used to image tissue calcium deposits in real-time during a medical procedure, extremely fast processing may be desirable. In situations where processing speed may be less important, slower more economical CPUs may be acceptable. Additionally, in some aspects a given system can have different CPUs in different SBCs. Different SBCs in a system may have different processing needs and can, therefore, utilized different CPUs. This is similarly true for other components of the SBCs. In other aspects, all SBCs in a system can utilize the same or similar CPUs regardless of differential SBC processing. Non-limiting examples of current CPUs includes Pentium processors, Core i3, i5 and i7 processors, Xeon processors, AMD FX, A-Series, Phenom II, and Athlon II processors, and the like.

Additionally, in other aspects it is contemplated that computational tasks can be accomplished using one or more GPUs. For example, in one aspect the processing of the transmission data from an image scanning procedure can be performed on a GPU device or GPU technology. In another aspect, the processing of the reflection data from an image scanning procedure can be performed on a GPU device. In other aspects, both the transmission and reflection processing can be performed on one or more GPU devices. As such it is considered to be within the present scope to perform any portion of the computation and data processing that can be processed via GPU technology on a GPU device. Moving such computation from CPU processing to GPU processing can dramatically increase the speed of intensive calculations, such as the transmission and reflection calculations, in some cases by a factor of 5, 10 or more. Accordingly, the above described node configurations can additionally include GPU devices where appropriate.

Regarding the GPU device hardware, it is contemplated that any GPU device that is capable of such data processing is considered to be within the present scope. Non-limiting examples of off the shelf GPU devices can include NVidia Tesla K10, K20, K20X, K40, and the like. GPUs can be programed by various programing platforms, including, for example, NVidia's CUDA technology. It is also contemplated that the GPU technology is not limiting, and that future generations of GPUs are considered to be within the present scope.

An exemplary Data Acquisition Node can include the SBC, FCHA, Data Acquisition (DA) cards, Mux cards, Waveform Generator (WFG) card, the CompactPCl backplane that the cards plug into and a 19-inch rack mount chassis. The chassis provides power and cooling to the backplane and also provides a high-voltage power source for the Mux cards. One of the Data Acquisition Nodes can be designated as a master. The Master Data Acquisition Node can include a Motion Control card in addition to the cards found in the Data Acquisition Node. The Motion Control card can be used to control the motion of the transducer array(s).

The Mux card is the interface between the transducer array and the DA Card. A high density coax cable assembly can connect the Mux card to the transducer array, although other comparable connections are contemplated. One example of a Mux card can accept 256 analog inputs/outputs to/from the transducer array. Through inputs for the DA Card, the Mux card can select 16 of the 256 channels for amplification (70 db) and filtering. The 16 channels of conditioned analog data are then presented to the DA card via the CompactPCl backplane. One exemplary DA card has 16 14-bit A/D chips. Each A/D chip digitizes the analog data received from the Mux card at a rate of 33 million samples per second. Each A/D chip stores the converted data in a double-buffered First-In-First-Out (FIFO) for later storage in the SBC's memory or directly to the Fibre Channel Host Adapter.

The shared file system can be any data storage system capable of storing data from the various nodes of the system. Such can include any non-transitory computer readable media system having sufficient capacity and speed to be operational with the system. Non-limiting examples can include banks of disk drives, RAID arrays, cloud storage, and the like.

The architecture is configured to funnel data rapidly to the shared file system. The shared file system is available to store data and make data available for processing or other output use. As one example data can be processed from a first slice, or data from the first slice can be accessed for computation, while storing data from the second slice. The present architecture thus provides for efficient and flexible examination of a subject. In a medical setting for example, a technician can review the data or generated images while the system is scanning. In addition, the data or images can be provided to a physician immediately after an examination to quickly provide results to the subject. Furthermore, in many cases the present architecture can reduce the size and cost of the computing components.

The system can also include various other components, including for example, a power source, a high voltage source (e.g., for pumps and the like), a low voltage source (e.g., for sensors, control valves, and the like), computer(s), and drive array(s), etc. Many or all of the various components can be contained in a housing so that the system can be compact and self-contained. A terminal can be coupled to the computers and/or other components in the base by standard connections. The terminal can be remote from the base, or can be physically coupled thereto.

In some aspects, the platform can include a Waveform Generator (WFG) to generate a digitally-programmed waveform for use by the system. In one aspect, the WFG can function as the source of the signal used to excite the ultrasound transmission transducer array.

In some aspects, the platform can also include a beam former. The beam former can be used to generate a digitally-programmed waveform for use by the reflection system. In addition, the beam former can create a time variable gain control to vary the gain of the transducer array amplifiers. The Beam Former can also generate master timing signals used to synchronize the data acquisition subsystem. One example of a suitable beam former is the cQuest Cicada Subsystem by Cephasonics Corp.

It is noted that, in some aspects, the reflection data can be collected from each component/element of the receiver array, that is, without using the beamforming capability. Such raw data can then be synthesized in software to dynamically focus at different depths, or synthetically focused to yield a reflection image.

In one specific aspect, a QTUS scanning system having a rotating transducer array is provided. As is shown in FIG. 5, The system can include an imaging chamber 102 and a tri-arm 508 upon which two opposing ultrasound transducer arrays 504 and a reflection array 506 are mounted to allow the arrays to move in relation to a body part 502. The arrays rotate and move up and down with the tri-arm to generate a complete 3-D data set for the area of interest or even for the entire body part 502. The transmission data is multiplexed to DA cards, and transferred to an on board RAID, where a separate processor (e.g. GPU card enhanced) applies the inverse scattering algorithm, then uses the resulting speed of sound (SOS) and/or attenuation map in the refraction corrected technique discussed herein. The reflection data is acquired through an independent DA system that also beam-forms the data.

For further details of systems that can be utilized to at least partially implement the QTUS imaging technology described herein, see U.S. Pat. No. 8,366,617, which is incorporated herein by reference in its entirety.

It is additionally contemplated that various systems including software platforms can be implemented to provide varying degrees of functionality to the QTUS system. Such software platforms and associated architectures should not be seen as limiting, and other implementations that differ from the following are considered to be within the present scope. In some aspects a system can be implemented to merely drive the ultrasound imaging apparatus. In other aspects, a system can drive the ultrasound imaging apparatus and/or process and analyze data to derive QTUS image results. In other aspect, systems can be realized that integrate the QTUS imaging system with higher level information processing such as, for example, medical records at a medical facility. One example of a software system including such integration is shown in FIG. 14.

Regarding the creation of transmission images in general, further details can be found in U.S. Pat. No. 8,246,543, which is incorporated herein by reference in its entirety. While the creation of reflection images is described herein and is contained in various patent references described above, the following pseudo-code provides an extremely high level summary of one such non-limiting process:

```
For lview = 1 , nview do
    If (correct_refraction = ‘yes’) read in filtered speed image
    Otherwise set slowness(I,j,k) = 1. For all I,j,k
    Apply digital gain (read in support)
        Or set support identically = 0.
        For lprobe = 1,3 do
            For level = 1,nlevels do
                Read in data
                For each beam: j=1,nbeams
                    Compute Hilbert Transform → form analytic
                signal Enddo (beams)
                For each beam: j=1,nbeams; do
                    Compute ray path through breast
                    Apply digital gain appropriately
                    Interpolate energy to reflection grid (3D) based on
                    ray path (solution to eikonal equation)
                Enddo (beam)
            Enddo (level)
        Enddo (probe)
Enddo (view)
Store images by view
Blend images to get 360 degree compounding or ‘clock view’ images.
Post-process 360 compounded volume and/or clock view images.
```

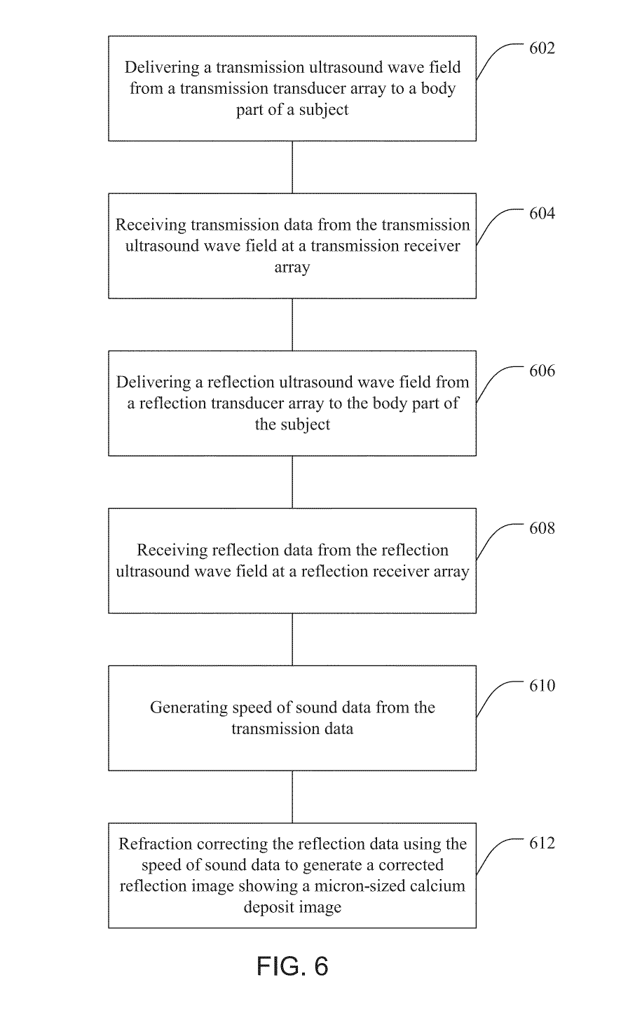

The present disclosure additionally provides methods for imaging calcium. In one specific aspect, a method for imaging a micron-sized calcium deposit in a tissue of a subject is provided, as is shown in FIG. 6. Such a method can include 602 delivering a transmission ultrasound wave field from a transmission transducer array to a body part of a subject, 604 receiving transmission data from the transmission ultrasound wave field at a transmission receiver array, 606 delivering a reflection ultrasound wave field from a reflection transducer array to the body part of the subject, and 608 receiving reflection data from the reflection ultrasound wave field at a reflection receiver array. The method can additionally include 610 generating speed of sound data from the transmission data, and 612 refraction correcting the reflection data using the speed of sound data to generate a corrected reflection image showing a micron-sized calcium deposit image.

It is noted that no specific order is to be implied by the method steps as they are presented above, and any potentially useful ordering of these steps is contemplated. In one aspect, for example, the transmission ultrasound wave field is delivered prior to the reflection ultrasound wave field. In another aspect, the reflection ultrasound wave field is delivered prior to the transmission ultrasound wave field. In yet another aspect, the transmission ultrasound wave field and the reflection ultrasound wave field are delivered simultaneously. In a further aspect, the transmission ultrasound wave field and the reflection ultrasound wave field are the same wave field.

The imaging methods can be performed a single time or multiple times. In one aspect, for example, the method can further include repositioning at least one of the transmission transducer array or the reflection transducer array, and repeating one or more of the steps of delivering the transmission ultrasound wave field, receiving the transmission data, delivering the reflection ultrasound wave field, and receiving the reflection data prior to refraction correcting the reflection data. In some aspects, the method can include repeating one or more of these steps at a plurality of repositioned locations.

One technique that allows the imaging of less than micron-sized calcium deposits includes the refraction correction of the reflection data. In one example, the reflection data can be refraction corrected directly as it is acquired, or following storage on a nontransitory computer readable medium. In other examples, the reflection data can be refraction corrected following one or more transformations of the acquired data. For example, in one aspect refraction correcting the reflection data can include generating an uncorrected reflection image from the reflection data and refraction correcting the uncorrected reflection image using the speed of sound data to generate the corrected reflection image.

In an exemplary embodiment, a method for imaging one or more micron-sized calcium deposits in a tissue of a subject can include:

delivering a transmission ultrasound wave field from a transmission transducer array to a body part of a subject;

receiving transmission data from the transmission ultrasound wave field at a transmission receiver array;

delivering a reflection ultrasound wave field from a reflection transducer array to the body part of the subject;

receiving reflection data from the reflection ultrasound wave field at a reflection receiver array;
generating speed of sound data from the transmission data; and refraction correcting the reflection data using the speed of sound data to generate a corrected reflection image showing a micron-sized calcium deposit image.

In another example embodiment, the transmission ultrasound wave field is delivered prior to the reflection ultrasound wave field.

In another example embodiment, the reflection ultrasound wave field is delivered prior to the transmission ultrasound wave field.

In another example embodiment, the transmission ultrasound wave field and the reflection ultrasound wave field are delivered simultaneously.

In another example embodiment, the transmission ultrasound wave field and the reflection ultrasound wave field are the same wave field.

In another example embodiment, the method can further include repositioning at least one of the transmission transducer array or the reflection transducer array, and repeating the steps of delivering the transmission ultrasound wave field, receiving the transmission data, delivering the reflection ultrasound wave field, and receiving the reflection data prior to refraction correcting the reflection data.

In another example embodiment, the steps of the previous example embodiment are repeated at a plurality of repositioned locations.

In another example embodiment, refraction correcting the reflection data further comprises:
generating an uncorrected reflection image from the reflection data; and generating the corrected reflection image by refraction correcting the uncorrected reflection image using the speed of sound data.

In another example embodiment, the calcium deposit image has an average size of from about 10 microns to about 1000 microns.

In another example embodiment, the calcium deposit image has an average size of from about 30 microns to about 500 microns.

In another example embodiment, the calcium deposit image is free of artifacts.

In another example embodiment, the artifacts of the previous example embodiment include multiple images of the same calcium deposit.

In another example embodiment, the method can further comprise attaching a coupling device to the body part to facilitate transmission of ultrasound energy to and from the body part.

In another example embodiment, the method can further comprise storing the transmission data and the reflection data in a nontransitory computer readable medium. In another example embodiment, the method can further comprise generating the speed of sound data and refraction correcting the reflection data using a computational processor functionally coupled to the nontransitory computer readable medium.

In another example embodiment, delivering reflection ultrasound further comprises:
delivering ultrasound energy having a fundamental frequency;

modeling a conversion of some acoustic energy to a second harmonic of the fundamental frequency; and receiving the second harmonic ultrasound energy.

In another example embodiment, the method can further include utilizing the corrected reflection image having the micron-sized calcium deposit image to diagnose a calcium-related condition.

In another example embodiment, the micron-sized calcium deposit is rendered as a 3-D volume of calcium.

In another example embodiment, an ultrasound scanning system for imaging micron-sized calcium deposits in a tissue of a subject comprises:

a transmission transducer array;

a transmission receiver array positioned to receive ultrasound energy transmitted through a body part and delivered from the transmission transducer array;

a reflection transducer array;

a reflection receiver array positioned to receive ultrasound energy reflected by the body part and delivered from the reflection transducer array; and

computation system functionally coupled to at least the transmission receiver array and to the reflection receiver array, and operable to generate speed of sound data from transmission data received by the transmission receiver array, and operable to process reflection data received by the reflection receiver array using the speed of sound data to generate a corrected reflection image.

In another example embodiment, the system can further comprise a beam former functionally coupled to at least one of the transmission transducer array or the reflection transducer array.

In another example embodiment, the computation system further comprises:
a nontransitory computer readable medium functionally coupled to the transmission receiver array and to the reflection receiver array, and operable to receive and store transmission data and reflection data; and

a computational module coupled to the nontransitory computer readable medium that is operable to generate speed of sound data from the transmission data, and to process the reflection data using the speed of sound data to generate a corrected reflection image.

In another example embodiment, the computational module is a computational processor.

In another example embodiment, the computational module further comprises:

a plurality of interconnected nodes including at least one compute node and at least one data acquisition node;

wherein the at least one compute node includes a single board computer and a fibre channel host adaptor, and the at least one data acquisition node includes a single board computer, a fibre channel host adaptor, a waveform generator card, at least one data acquisition card, and at least one Mux card.

### EXAMPLES

### Example 1

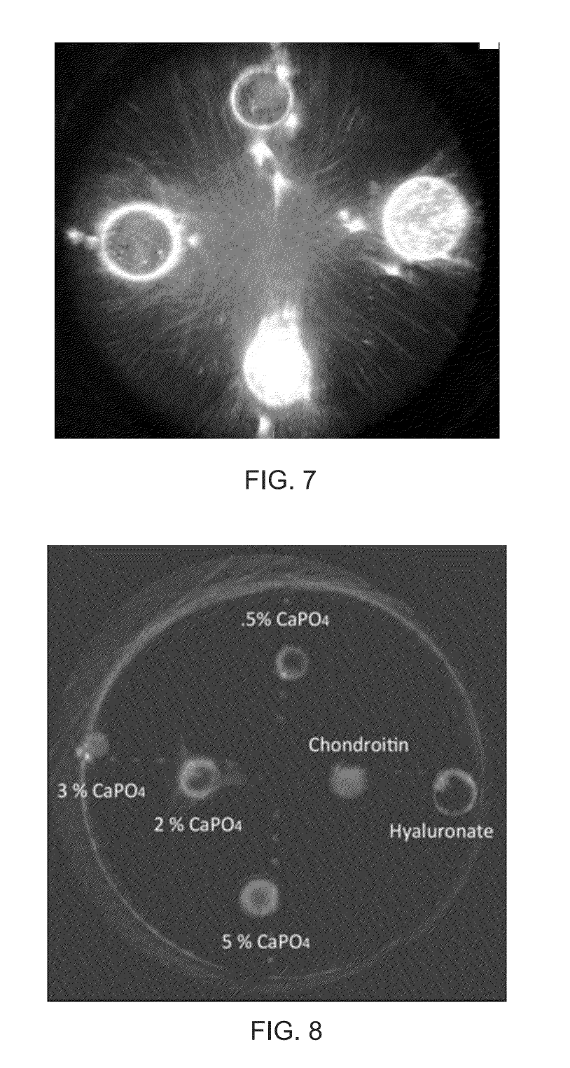

As is shown in FIG. 7, an image of balloon phantoms in tissue containing various amounts of calcium crystals was obtained using QTUS imaging.

### Example 2

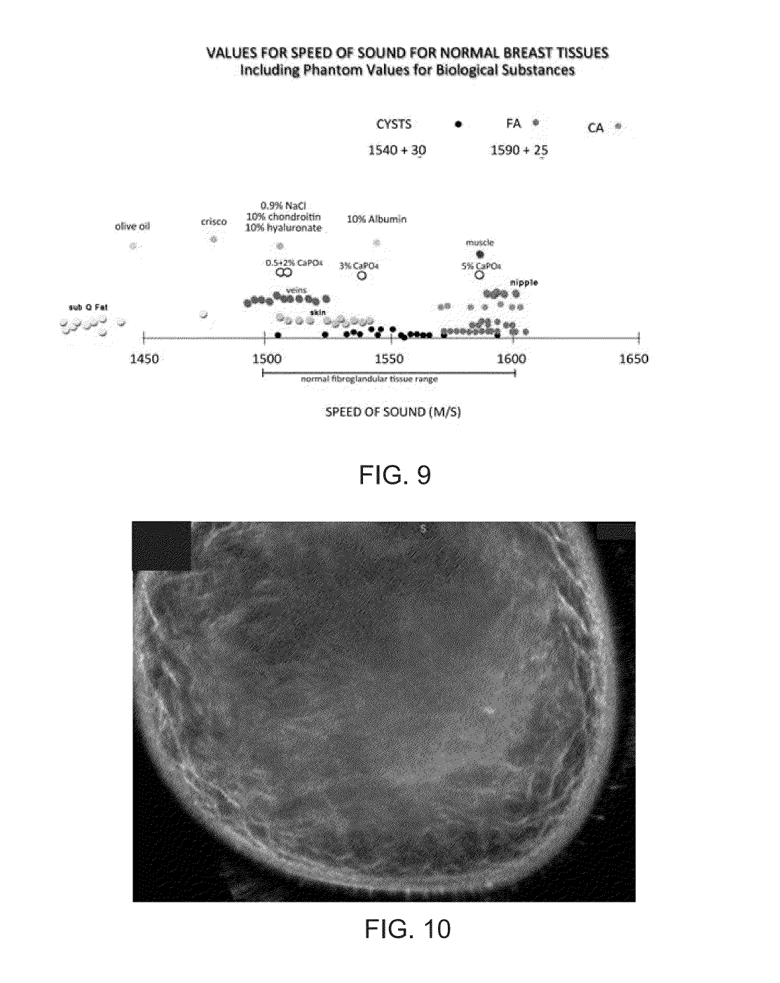

As is shown in FIG. 8, an image of balloon phantoms containing various amounts of calcium phosphate solutions was obtained using QTUS imaging. FIG. 9 shows a graph depicting different speeds of sound through different human breast tissue types obtained while QTUS imaging normal human and cadaver breasts, as well as phantom values for various biological substances.

### Example 3

As is shown in FIG. 10, an image of a human subject's breast showing a deposit of calcium was obtained using QTUS imaging.

### Example 4

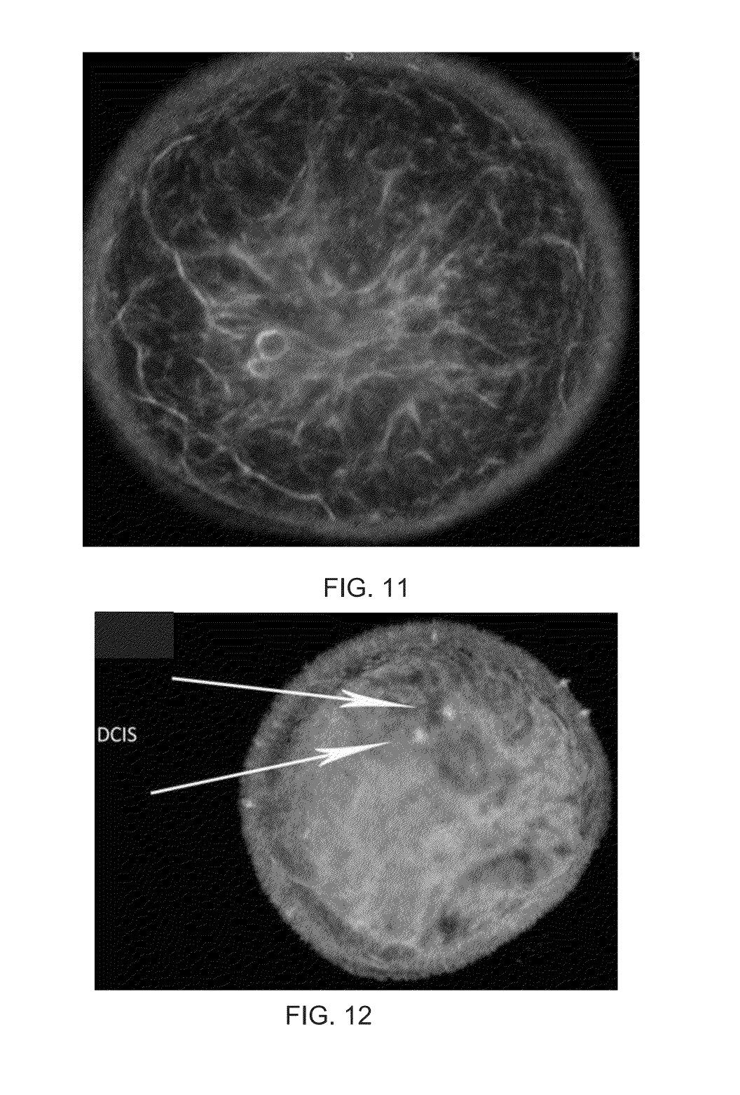

As is shown in FIG. 11, an image of a human subject's breast showing a deposit of calcium around the rim of several oil cysts was obtained using QTUS imaging

### Example 5

As is shown in FIG. 12, an image of a human subject's breast showing deposits of calcium in the breast diagnosed as ductal carcinoma in situ was obtained using QTUS imaging

### Example 6

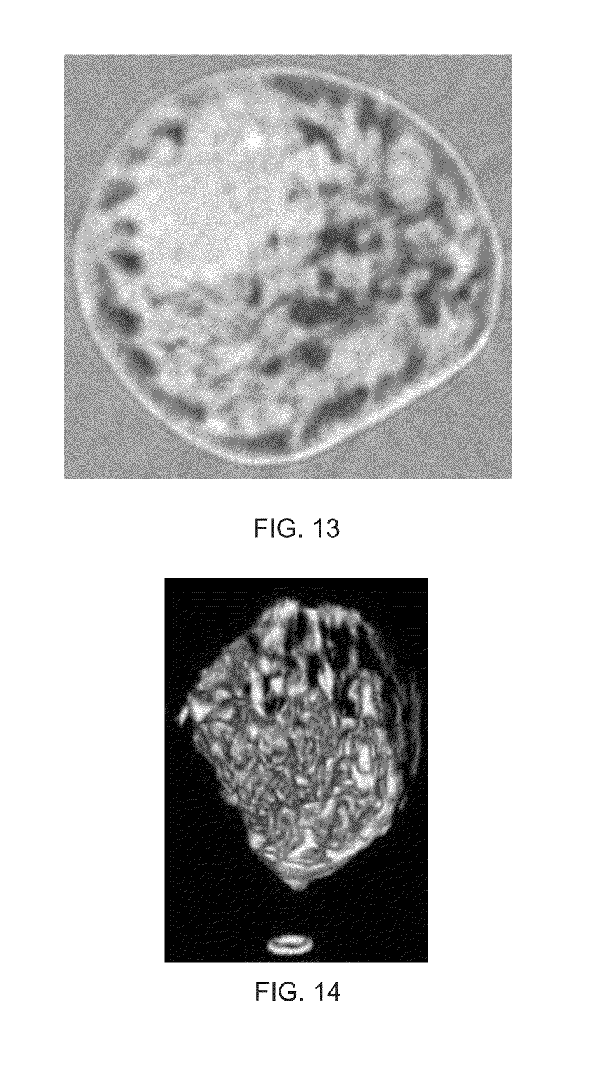

As is shown in FIG. 13, an image of a human subject's breast showing a large deposit of calcium in the breast was obtained using QTUS imaging

### Example 7

As is shown in FIG. 14, an image of results of using calcification parameters from the QTUS image of FIG. 13 to develop an interpretive image of the calcium of the breast of FIG. 10 was obtained. This information can be used to isolate the calcification and create a 3-D image of the breast calcium, including its location and size and shape.

It is to be understood that the above-described arrangements and examples are only illustrative of the application of the principles of the present disclosure. Numerous modifications and alternative arrangements may be devised by those skilled in the art without departing from the spirit and scope of the present disclosure, and the appended claims are intended to cover such modifications and arrangements. Thus, while the present disclosure has been described above with particularity and detail in connection with what is presently deemed to be the most practical embodiments of the disclosure, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A method for imaging a micron-sized calcium deposit in a tissue of a subject, comprising:
delivering a transmission ultrasound wave field from a transmission transducer array to a body part of a subject;
receiving transmission data from the transmission ultrasound wave field at a transmission receiver array;
delivering a reflection ultrasound wave field from a reflection transducer array to the body part of the subject;
receiving reflection data from the reflection ultrasound wave field at a reflection receiver array;
generating speed of sound data from the transmission data; and
refraction correcting the reflection data using the speed of sound data to generate a corrected reflection image showing a micron-sized calcium deposit image.

2. The method of claim 1, wherein the transmission ultrasound wave field is delivered prior to the reflection ultrasound wave field.

3. The method of claim 1, wherein the reflection ultrasound wave field is delivered prior to the transmission ultrasound wave field.

4. The method of claim 1, wherein the transmission ultrasound wave field and the reflection ultrasound wave field are delivered simultaneously.

5. The method of claim 4, wherein the transmission ultrasound wave field and the reflection ultrasound wave field are the same wave field.

6. The method of claim 1, further comprising:
repositioning at least one of the transmission transducer array or the reflection transducer array; and
repeating the steps of delivering the transmission ultrasound wave field, receiving the transmission data, delivering the reflection ultrasound wave field, and receiving the reflection data prior to refraction correcting the reflection data.

7. The method of claim 6, further comprising repeating the steps of claim 6 at a plurality of repositioned locations.

8. The method of claim 1, wherein refraction correcting the reflection data further comprises:
generating an uncorrected reflection image from the reflection data; and
generating the corrected reflection image by refraction correcting the uncorrected reflection image using the speed of sound data.

9. The method of claim 1, wherein the calcium deposit image has an average size of from about 10 microns to about 1000 microns.

10. The method of claim 1, wherein the calcium deposit image has an average size of from about 30 microns to about 500 microns.

11. The method of claim 1, wherein the calcium deposit image is free of artifacts.

12. The method of claim 11, wherein the artifacts include multiple images of the same calcium deposit.

13. The method of claim 1, further comprising attaching a coupling device to the body part to facilitate transmission of ultrasound energy to and from the body part.

14. The method of claim 1, further comprising storing the transmission data and the reflection data in a nontransitory computer readable medium.

15. The method of claim 14, further comprising generating the speed of sound data and refraction correcting the reflection data using a computational processor functionally coupled to the nontransitory computer readable medium.

16. The method of claim 1, wherein delivering reflection ultrasound further comprises:
delivering ultrasound energy having a fundamental frequency;
modeling a conversion of some acoustic energy to a second harmonic of the fundamental frequency; and
receiving the second harmonic ultrasound energy.

17. The method of claim 1, further comprising utilizing the corrected reflection image having the micron-sized calcium deposit image to diagnose a calcium-related condition.

18. The method of claim 1, wherein the micron-sized calcium deposit is rendered as a 3-D volume of calcium.

19. An ultrasound scanning system for imaging micron-sized calcium deposits in a tissue of a subject, comprising:
a transmission transducer array;
a transmission receiver array positioned to receive ultrasound energy transmitted through a body part and delivered from the transmission transducer array;
a reflection transducer array;
a reflection receiver array positioned to receive ultrasound energy reflected by the body part and delivered from the reflection transducer array; and
computation system functionally coupled to at least the transmission receiver array and to the reflection receiver array, and operable to generate speed of sound data from transmission data received by the transmission receiver array, and operable to process reflection data received by the reflection receiver array using the speed of sound data to generate a corrected reflection image.

20. The system of claim 19, further comprising a beam former functionally coupled to at least one of the transmission transducer array or the reflection transducer array.

21. The system of claim 19, where in the computation system further comprises:
a nontransitory computer readable medium functionally coupled to the transmission receiver array and to the reflection receiver array, and operable to receive and store transmission data and reflection data; and
a computational module coupled to the nontransitory computer readable medium that is operable to generate speed of sound data from the transmission data, and to process the reflection data using the speed of sound data to generate a corrected reflection image.

22. The system of claim 21, wherein the computational module is a computational processor.

23. The system of claim 22, wherein the computational module further comprises:
a plurality of interconnected nodes including at least one compute node and at least one data acquisition node;
wherein the at least one compute node includes a single board computer and a fibre channel host adaptor, and the at least one data acquisition node includes a single board computer, a fibre channel host adaptor, a waveform generator card, at least one data acquisition card, and at least one Mux card.
